(19) United States
(12) Patent Application Publication
(10) Pub. No.: US 2017/0076047 A1
(43) Pub. Date: Mar. 16, 2017

(54) SYSTEMS AND METHODS FOR GENETIC TESTING

(71) Applicant: Good Start Genetics, Inc., Cambridge, MA (US)

(72) Inventor: Mark Adams, Cambridge, MA (US)

(21) Appl. No.: 15/266,717

(22) Filed: Sep. 15, 2016

Related U.S. Application Data
(60) Provisional application No. 62/219,408, filed on Sep. 16, 2015.

(51) Int. Cl. G06F 19/00; A61B 5/00; G06F 17/30

(52) U.S. Cl. CPC G06F 19/322; G06F 19/345; G06F 17/30979; G06F 17/30991; A61B 5/746; A61B 5/0075; A61B 5/0071

(57) ABSTRACT

A genetic analysis system that provides a notification of new medical information that is non-trivial and significant to the results of a patient's prior genetic test. The system retrieves clinical information from an outside database and also evaluates whether subsequent updates to that database are significant to the patient. If significant, the system provides a notification of the availability of new clinical information. Methods of the invention includes obtaining sequence data for a patient, retrieving from a database clinical information on a variant in the sequence data, and associating the clinical information with the variant in the memory subsystem. The method further includes determining whether an update to the clinical information has been published, evaluating significance of the update, and notifying a user of updated clinical information when significant.

### CROSS-REFERENCE TO RELATED APPLICATIONS

This application claims priority to, and the benefit of, U.S. Provisional Application No. 62/219,408, filed Sep. 16, 2015, which is incorporated by reference in its entirety.

### TECHNICAL FIELD

The invention relates to medical genetics.

### BACKGROUND

Some babies are born with genetic disorders such as cystic fibrosis, Tay-Sachs, or hemophilia. Such disorders are problems caused by abnormalities in the genome and in many cases can be detected by genetic testing methods such as by sequencing DNA. Studying a person's genes and any abnormalities therein provide doctors with important tools for managing and treating the genetic disorders and their symptoms. Unfortunately, providing effective treatment for a patient with a genetic disorder is not always so simple as sequencing their genome and looking up the results.

Human genetics is a technical field that continues to make advances. New mutations are discovered, new relationships among mutations are discovered, and new links between mutations and diseases are established as researchers make progress. A patient whose genes are screened by sequencing may be provided with a report that gives genetic information. Some variants may be listed in the report as associated with a condition or some may be marked as variant of unknown significance. But a patient will not know when researchers have gleaned new information. Even if a patient were to seek further information after a genetic test, the sheer volume of new information (re-classifications, updated accession numbers, new disease information, clinical trial reports of minor significance, literature reviews with no real impact on that patient) would flood the patient with an insolubly dense library of raw data.

### SUMMARY

The invention provides a system for genetic analysis that provides a notification of new medical information that is non-trivial and significant to the results of a patient's prior genetic test. The system can be used for analyzing sequencing data to identify mutations and composing a patient report that includes the patient's genetic information as well as clinical information relevant to the identified mutations. The system pulls at least some of the clinical information from an outside database such as a third-party clinical decision support resource. When the outside database is updated, the system evaluates whether the new information rises to a certain level of significance to that patient with that mutation and, if so, can notify a user such as the patient's physician or genetic counselor of the new clinical information. The system can produce a new report for the patient that includes the new information or an action plan based on the new information. The evaluation of the significance of the new information can take into account both the scope of the change and the impact to the particular patient. Thus some updates may be deemed trivial and ignored, for example, where a minor change is documented in incidence of a disease in some demographic. Additionally, updates need not trigger a notification if not relevant to the patient, for example, a where a SNP is linked to prostate cancer a female patient may not be given an urgent notification.

Since the system evaluates the updates that are made in the outside database for scope and impact, the patient or the patient's care provider receives notifications when updates are made that will be informative to the patient and will not be notified of each and every mention of a gene or mutation in the medical literature. Since the system can operate to automatically query the outside database in real time, the patient can learn the new medical information as soon as it is curated for inclusion in the medical literature or outside database. Since patients receive new medical information promptly, not only are their opportunities for treatment greatly improved, but less useful or dated understandings are superseded as fast as the new innovations in medical genetics are made. Since patients with genetic conditions are guided to the most up-to-date clinical information as it becomes available, lives may be saved and people's quality of life may be greatly improved.

In certain aspects, the invention provides a method for updating informative content of genomic information. The method includes obtaining sequence data from a sample from a patient, inputting the sequence data into a computer system, retrieving from a database clinical information on at least one variant in the sequence data, and associating the clinical information with the variant in the memory subsystem. Continuing to use the computer system, the method further includes determining whether an update to the clinical information has been published, evaluating whether the update meets predetermined criteria for significance, and notifying a user of updated clinical information meeting the predetermined criteria for significance. The sequence data may be obtained by sequencing nucleic acid from the sample to obtain a plurality of sequence reads. The sequence reads may be mapped to a genomic reference to identify the at least one variant and the at least one variant is stored in a memory subsystem as a variant call prior to retrieving the information on the variant.

In some embodiments, the database is a curated database on a remote computer system. The evaluating step may include reading metadata entered into the database. The metadata identifies a source of the update, a date of the update, the predetermined criteria, or such.

In certain embodiments, the method includes providing a report for a user that includes an identity of the patient, the variant call, and the clinical information on the variant, and later providing an updated reported with the updated clinical information. Preferably, the clinical information associated with the variant in a memory subsystem of the computer system includes one or more of a functional information, a disease association, and medical information. A report provided to a user prior to the determining step may include the clinical information associated with the variant and an identity of the patient. Optionally, the determining, evaluating, and notifying steps are performed a plurality of times for a plurality of different updates over a period of at least a week. Preferably, the clinical information includes one or more of an association of a variant in the sequence data with a medical condition, a prognosis, a treatment regimen, or a propensity for disease. At least a portion of the computer system may be provided by a cloud-based system in which another processor may be substituted for the processor without interfering with operation of the method. The method may include notifying the user of the updated clinical information comprises sending an alert from the computer system to a user computer device (e.g., causing the alert to be displayed on a mobile or web interface on the user computer device). In the certain embodiments, obtaining the sequence data comprises sequencing nucleic acid from the sample to obtain a plurality of sequence reads, and the may include mapping the sequence reads to a genomic reference to identify the at least one variant, storing the at least one variant in the memory subsystem as a variant call prior to retrieving the information on the variant, providing a report for a user that includes an identity of the patient, the variant call, and the clinical information on the variant, and later providing an updated reported with the updated clinical information.

Aspects of the invention provide a system for updating informative content of genomic information. The system includes a processor coupled to a tangible memory subsystem storing instructions that when executed by the processor cause the system to obtain sequence data from a sample from a patient, retrieve from a database clinical information on at least one variant in the sequence data, and associate the clinical information with the variant in the memory subsystem. Further, the system will determine whether an update to the clinical information has been published, evaluate whether the update meets predetermined criteria for significance, and notify a user of updated clinical information meeting the predetermined criteria for significance. Preferably, the database is a curated database on a remote computer system.

The evaluating step may include reading metadata entered into said database. The metadata may identify at least one of a source of the update, a date of the update, and the predetermined criteria. The system may be further operable to map the sequence data to a genomic reference to identify the at least one variant and store the at least one variant in the memory subsystem as a variant call prior to retrieving the information on the variant. Additionally or alternatively, the system is further operable to provide a report for a user that includes an identity of the patient, the variant call, and the clinical information on the variant, and later provide an updated reported with the updated clinical information. The clinical information associated with the variant in the memory subsystem can include one or more of a functional information, a disease association, and medical information. In some embodiments, the system is operable to provide a report to a user prior to determining whether the update has been published, wherein the report includes the clinical information associated with the variant (e.g., an association of a variant in the sequence data with a medical condition, a prognosis, a treatment regimen, or a propensity for disease) and an identity of the patient.

### BRIEF DESCRIPTION OF THE DRAWINGS

FIG. 1 diagrams a method for providing updated clinical information.

FIG. 2 illustrates use of MIPs to capture regions of target genomic material.

FIG. 3 gives a diagram of a workflow for variant detection.

FIG. 4 illustrates a platform architecture for implementing methods of the invention.

FIG. 5 gives a diagram of a system of the invention.

FIG. 6 diagrams a workflow for the medical information.

FIG. 7 shows determining whether to notify a user of the availability of a report.

FIG. 8 is a flowchart for determining a significance of an update.

### DETAILED DESCRIPTION

A genetic analysis system that provides a notification of new medical information that is non-trivial and significant to the results of a patient's prior genetic test. The system retrieves clinical information from an outside database and also evaluates whether subsequent updates to that database are significant to the patient. If significant, the system provides a notification of the availability of new clinical information. Methods of the invention includes obtaining sequence data for a patient, retrieving from a database clinical information on a variant in the sequence data, and associating the clinical information with the variant in the memory subsystem. The method further includes determining whether an update to the clinical information has been published, evaluating significance of the update, and notifying a user of updated clinical information when significant.

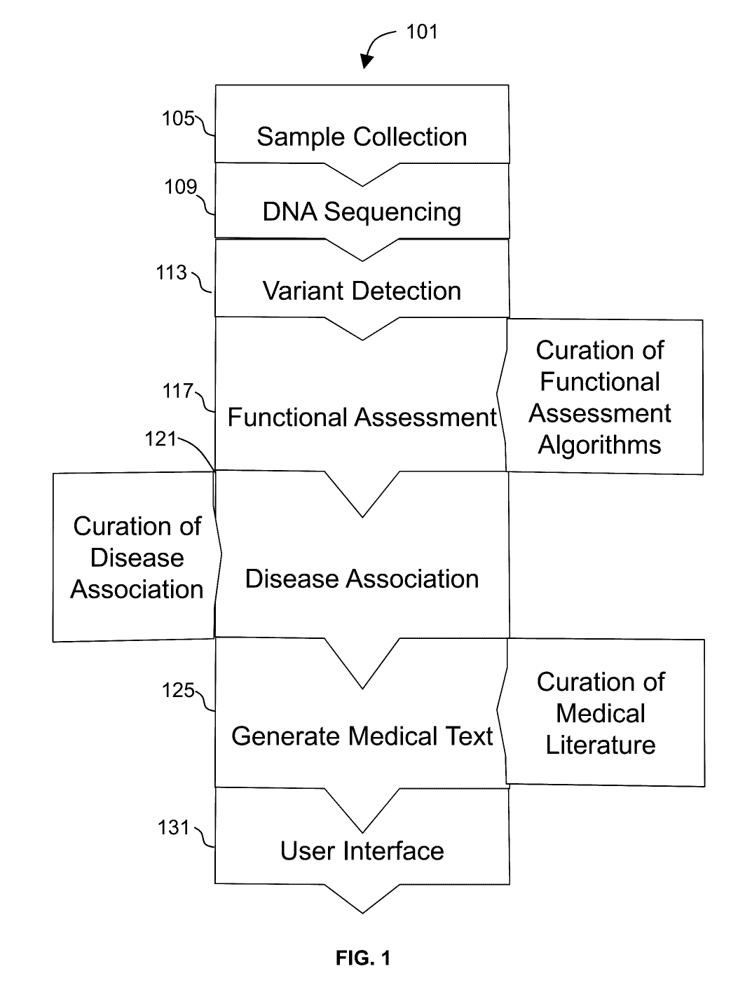

FIG. 1 diagrams a method 101 for providing updated clinical information. Sample collection 105 may include collecting saliva samples. Samples may be collected using a custom kit such as kit that a patient or a patient's parent orders from a website/mobile app. In some embodiments, parent take a cheek swab from themselves or a child and send the sample to a clinical facility. Data about the patient may be added at the time of collection to the desktop or mobile app. The sample is sequenced 109 according to sample prep and sequencing methods described herein. Data may be uploaded to an analysis platform (e.g., AWS S3) in near-real-time for processing and analysis. Data may be placed for permanent storage (e.g., Amazon Glacier). Variant detection 113 may proceed by any suitable method. For example, variant detection may include methods described herein and may employ tools such as cpipe or GATK. The variant detection operation 113 describes single nucleotide variation (SNV), substitutions, and insertion or deletion variants (indels) across the patient's exome or genome. Functional assessment 117 may be performed to assess a functional significance of a mutation. Functional assessment 117 may use such tools such as Genospace, Broad Inst., Signifikance, etc., to assess a functional impact of a variant through the application of a range of algorithmic and heuristic approaches. Methods of the invention provide for the ongoing, agent-based update and analysis of all variant data in the system. Curated updates to detection algorithms trigger agent-based database updates. In disease association 121, variants may be associated with a disease and any additional information. This may be performed through the systematic and semantically-controlled combination of manual and automated curation, leveraging a complete range of public and private data sources. A customized curation workbench facilitates the curation process. Systems and methods of the invention are operable to generate medical text 125. The medical text can be provided by querying an outside source such as a clinical decision support resource as the outside database. One suitable product is the clinical decision support resource offered under the trademark UP2DATE by Wolters-Kluwer. Systems and methods of the invention use automated access to structured, actionable medical information for specific diseases from the outside database and provide for custom integration of updates based on new “tagged content” from the outside database. The outside databases may customize medical content for parents and pediatricians and be queried for medical information by mutation or variant. Systems and methods of the invention provide a user interface 131 such as a mobile app and desktop web app to provide personalized access to updated data. Alerts generated by curated updates of relevant information are automatically pushed out to applicable patients, parents, doctors, or genetic counselors.

### 1. Sample Collection & Sample Prep

Sample collection 105 may include collecting saliva samples. Samples may be collected using a custom kit. Kits may be ordered from the website/mobile app. In some embodiments, parents collect and send the sample themselves. Data about the child and parents added at the time of collection to the desktop or mobile app. Additionally or alternatively, a sample may be obtained from a tissue or body fluid that is obtained in any clinically acceptable manner. Body fluids may include mucous, blood, plasma, serum, serum derivatives, bile, blood, maternal blood, phlegm, saliva, sweat, amniotic fluid, menstrual fluid, mammary fluid, follicular fluid of the ovary, fallopian tube fluid, peritoneal fluid, urine, and cerebrospinal fluid (CSF), such as lumbar or ventricular CSF. A sample may also be a fine needle aspirate or biopsied tissue. A sample also may be media containing cells or biological material. Samples may also be obtained from the environment (e.g., air, agricultural, water and soil) or may include research samples (e.g., products of a nucleic acid amplification reaction, or purified genomic DNA, RNA, proteins, etc.).

Isolation, extraction or derivation of genomic nucleic acids may be performed by methods known in the art. Isolating nucleic acid from a biological sample generally includes treating a biological sample in such a manner that genomic nucleic acids present in the sample are extracted and made available for analysis. Generally, nucleic acids are extracted using techniques such as those described in Green & Sambrook, 2012, Molecular Cloning: A Laboratory Manual 4 edition, Cold Spring Harbor Laboratory Press, Cold Spring Harbor, N.Y. (2028 pages), the contents of which are incorporated by reference herein. A kit may be used to extract DNA from tissues and bodily fluids and certain such kits are commercially available from, for example, BD Biosciences Clontech (Palo Alto, Calif.), Epicentre Technologies (Madison, Wis.), Gentra Systems, Inc. (Minneapolis, Minn.), and Qiagen Inc. (Valencia, Calif.). User guides that describe protocols are usually included in such kits.

It may be preferable to lyse cells to isolate genomic nucleic acid. Cellular extracts can be subjected to other steps to drive nucleic acid isolation toward completion by, e.g., differential precipitation, column chromatography, extraction with organic solvents, filtration, centrifugation, others, or any combination thereof. The genomic nucleic acid may be re-suspended in a solution or buffer such as water, Tris buffers, or other buffers. In certain embodiments the genomic nucleic acid can be re-suspended in Qiagen DNA hydration solution, or other Tris-based buffer of a pH of around 7.5. Isolated nucleic acid (e.g., DNA, RNA, cDNA, etc.) may be fragmented for enhanced probe capture. Methods of nucleic acid fragmentation are known in the art and include, but are not limited to, DNase digestion, sonication, mechanical shearing, and the like. U.S. Pub 2005/0112590 provides a general overview of various methods of fragmenting known in the art. Fragmentation of nucleic acid target is discussed in U.S. Pub. 2013/0274146. The nucleic acid can also be sheared via nebulization, hydro-shearing, sonication, or others. See U.S. Pat. No. 6,719,449; U.S. Pat. No. 6,948,843; and U.S. Pat. No. 6,235,501. In certain embodiments, the sample nucleic acid is captured or targeted using any suitable capture method or assay such as hybridization capture or capture by probes such as one or more of a molecular inversion probe (MIP).

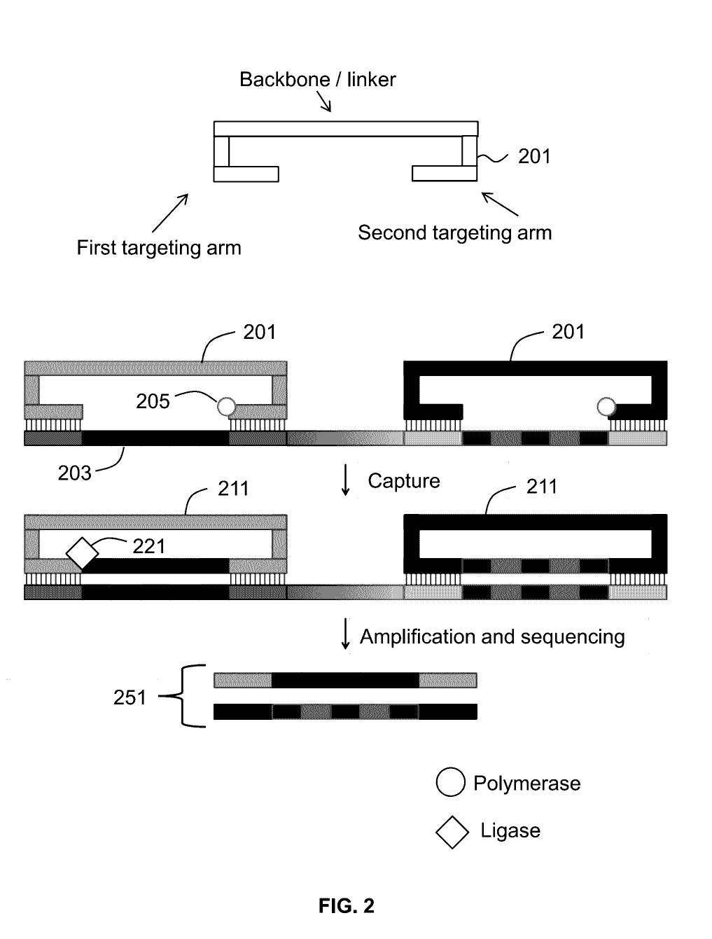

FIG. 2 illustrates use of MIPs 201 to capture regions of target genomic material 203 for amplification and sequencing. Each MIP 201 contains a common backbone sequence and two complementary arms that are annealed to a DNA sample of interest. A polymerase 205 is utilized to fill in the gap between each of the two arms, and a ligase 221 is then utilized to create a set of circular molecules. Capture efficiency of the MIP to the target sequence on the nucleic acid fragment can be optimized by lengthening the hybridization and gap-filing incubation periods. (See, e.g., Turner et al., 2009, Massively parallel exon capture and library-free resequencing across 16 genomes, Nature Methods 6:315-316.) The resultant circular molecules 211 can be amplified using polymerase chain reaction to generate a targeted sequencing library.

MIPs can be used to detect or amplify particular nucleic acid sequences in complex mixtures. Use of molecular inversion probes has been demonstrated for detection of single nucleotide polymorphisms (Hardenbol et al., 2005, Highly multiplexed molecular inversion probe genotyping: over 10,000 targeted SNPs genotyped in a single tube assay, Genome Res 15:269-75) and for preparative amplification of large sets of exons (Porreca et al., 2007, Multiplex amplification of large sets of human exons, Nat Methods 4:931-6 and Krishnakumar et al., 2008, A comprehensive assay for targeted multiplex amplification of human DNA sequences, PNAS 105:9296-301). One significant benefit of the method is in its capacity for a high degree of multiplexing, because generally thousands of targets may be captured in a single reaction containing thousands of probes.

In some embodiments, the amount of target nucleic acid and probe used for each reaction is normalized to avoid any observed differences being caused by differences in concentrations or ratios. In some embodiments, in order to normalize genomic DNA and probe, the genomic DNA concentration is read using a standard spectrophotometer or by fluorescence (e.g., using a fluorescent intercalating dye). The probe concentration may be determined experimentally or using information specified by the probe manufacturer.

Once a locus has been captured, it may be amplified and/or sequenced in a reaction involving one or more primers. The amount of primer added for each reaction can range from 0.1 pmol to 1 nmol, 0.15 pmol to 1.5 nmol (for example around 1.5 pmol). However, other amounts (e.g., lower, higher, or intermediate amounts) may be used.

A targeting arm may be designed to hybridize (e.g., be complementary) to either strand of a genetic locus of interest of the nucleic acid being analyzed. For MIP probes, whichever strand is selected for one targeting arm will be used for the other one. It also should be appreciated that MIP probes referred to herein as “capturing” a target sequence are actually capturing it by template-based synthesis rather than by capturing the actual target molecule (other than for example in the initial stage when the arms hybridize to it or in the sense that the target molecule can remain bound to the extended MIP product until it is denatured or otherwise removed). Other MIP capture techniques are shown in U.S. Pub. 2012/0165202, incorporated by reference.

Multiple probes, e.g., MIPs, can be used to amplify each target nucleic acid. In some embodiments, the set of probes for a given target can be designed to ‘tile’ across the target, capturing the target as a series of shorter sub targets. In some embodiments, where a set of probes for a given target is designed to ‘tile’ across the target, some probes in the set capture flanking non-target sequence. Alternately, the set can be designed to ‘stagger’ the exact positions of the hybridization regions flanking the target, capturing the full target (and in some cases capturing flanking non-target sequence) with multiple probes having different targeting arms, obviating the need for tiling. The particular approach chosen will depend on the nature of the target set. For example, if small regions are to be captured, a staggered-end approach might be appropriate, whereas if longer regions are desired, tiling might be chosen. In all cases, the amount of bias-tolerance for probes targeting pathological loci can be adjusted by changing the number of different MIPs used to capture a given molecule. Probes for MIP capture reactions may be synthesized on programmable microarrays to provide the large number of sequences required. See e.g., Porreca et al., 2007, Multiplex amplification of large sets of human exons, Nat Meth 4(11):931-936; Garber, 2008, Fixing the front end, Nat Biotech 26(10):1101-1104; Turner et al., 2009, Methods for genomic partitioning, Ann Rev Hum Gen 10:263-284; and Umbarger et al., 2014, Next-generation carrier screening, Gen Med 16(2):132-140. Using methods described herein, a single copy of a specific target nucleic acid may be amplified to a level that can be sequenced. Further, the amplified segments created by an amplification process such as PCR may be, themselves, efficient templates for subsequent PCR amplifications.

The result of MIP capture as described in FIG. 2 includes one or more circular target probes, which then can be processed in a variety of ways. Adaptors for sequencing may be attached during common linker-mediated PCR, resulting in a library with non-random, fixed starting points for sequencing. For preparation of a shotgun library, a common linker-mediated PCR is performed on the circle target probes, and the post-capture amplicons are linearly concatenated, sheared, and attached to adaptors for sequencing. Methods may include attachment of amplification or sequencing adaptors or barcodes or a combination thereof to target DNA captured by probes.

Amplification or sequencing adapters or barcodes, or a combination thereof, may be attached to the fragmented nucleic acid. Such molecules may be commercially obtained, such as from Integrated DNA Technologies (Coralville, Iowa). In certain embodiments, such sequences are attached to the template nucleic acid molecule with an enzyme such as a ligase. Suitable ligases include T4 DNA ligase and T4 RNA ligase, available commercially from New England Biolabs (Ipswich, Mass.). The ligation may be blunt ended or via use of complementary overhanging ends.

In certain embodiments, one or more barcodes is or are attached to each, any, or all of the fragments. A barcode sequence generally includes certain features that make the sequence useful in sequencing reactions. The barcode sequences are designed such that each sequence is correlated to a particular portion of nucleic acid, allowing sequence reads to be correlated back to the portion from which they came. Methods of designing sets of barcode sequences is shown for example in U.S. Pat. No. 6,235,475, the contents of which are incorporated by reference herein in their entirety. In certain embodiments, the barcode sequences range from about 5 nucleotides to about 15 nucleotides. In a particular embodiment, the barcode sequences range from about 4 nucleotides to about 7 nucleotides. In certain embodiments, the barcode sequences are attached to the template nucleic acid molecule, e.g., with an enzyme. The enzyme may be a ligase or a polymerase, as discussed above. Attaching bar code sequences to nucleic acid templates is shown in U.S. Pub. 2008/0081330 and U.S. Pub. 2011/0301042, the content of each of which is incorporated by reference herein in its entirety. Methods for designing sets of bar code sequences and other methods for attaching barcode sequences are shown in U.S. Pat. Nos. 7,537,897; 6,138,077; 6,352,828; 5,636,400; 6,172,214; and 5,863,722, the content of each of which is incorporated by reference herein in its entirety. After any processing steps (e.g., obtaining, isolating, fragmenting, amplification, or barcoding), nucleic acid can be sequenced.

### 3. Sequencing

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

A sequencing technique that can be used includes, for example, Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. No. 7,960,120; U.S. Pat. No. 7,835,871; U.S. Pat. No. 7,232,656; U.S. Pat. No. 7,598,035; U.S. Pat. No. 6,911,345; U.S. Pat. No. 6,833,246; U.S. Pat. No. 6,828,100; U.S. Pat. No. 6,306,597; U.S. Pat. No. 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing produces a plurality of sequence reads. Reads generally include sequences of nucleotide data wherein read length may be associated with sequencing technology. For example, the single-molecule real-time (SMRT) sequencing technology of Pacific Bio produces reads thousands of base-pairs in length. For 454 pyrosequencing, read length may be about 700 bp in length. In some embodiments, reads are less than about 500 bases in length, or less than about 150 bases in length, or less than about 90 bases in length. In certain embodiments, reads are between about 80 and about 90 bases, e.g., about 85 bases in length. In some embodiments, these are very short reads, i.e., less than about 50 or about 30 bases in length. Sequence reads 251 can be analyzed to detect and describe the deletion 303 in target nucleic acid 203.

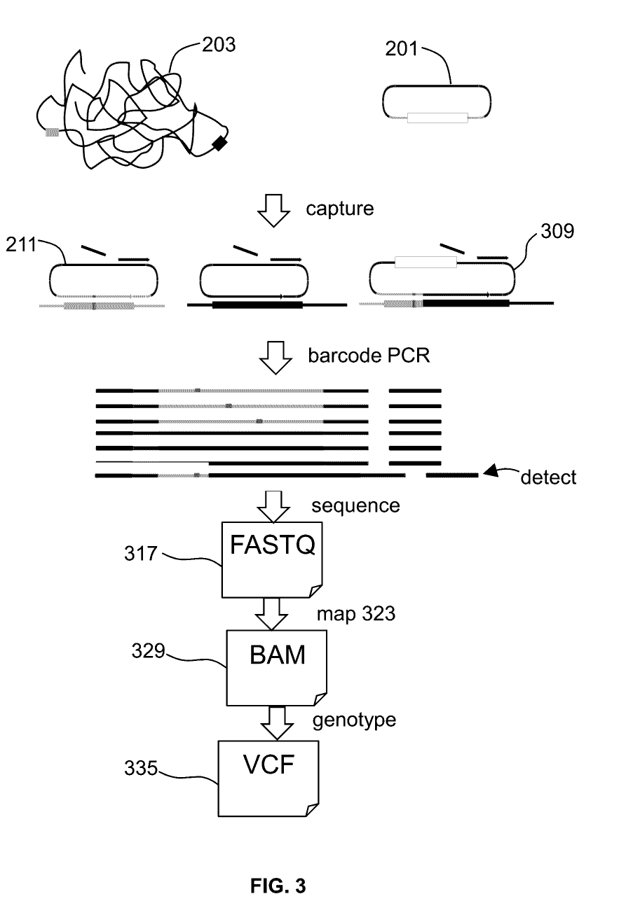

FIG. 3 gives a diagram of a workflow for variant detection 113. Genomic DNA 203 is used as a starting sample and is exposed to a plurality of MIPs 201. Hybridization of the MIPs provides circularized probe product 211. Barcode PCR may be performed to provide amplicon material for sequencing. The amplicons may then be sequenced. Sequencing produces a plurality of sequence reads.

Sequence read data can be stored in any suitable file format including, for example, VCF files, FASTA files or FASTQ files, as are known to those of skill in the art. In some embodiments, PCR product is pooled and sequenced (e.g., on an Illumina HiSeq 2000). Raw .bcl files are converted to qseq files using bclConverter (Illumina). FASTQ files are generated by “de-barcoding” genomic reads using the associated barcode reads; reads for which barcodes yield no exact match to an expected barcode, or contain one or more low-quality base calls, may be discarded. Reads may be stored in any suitable format such as, for example, FASTA or FASTQ format.

FASTA is originally a computer program for searching sequence databases and the name FASTA has come to also refer to a standard file format. See Pearson & Lipman, 1988, Improved tools for biological sequence comparison, PNAS 85:2444-2448. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There should be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer. Cock et al., 2009, The Sanger FASTQ file format for sequences with quality scores, and the Solexa/Illumina FASTQ variants, Nucleic Acids Res 38(6):1767-1771.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with “-”. In a preferred embodiment, the sequence data will use the A, T, C, G, and N characters, optionally including “-” or U as-needed (e.g., to represent gaps or uracil, respectively).

Following sequencing, reads may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated herein by reference. Strategies may include (i) assembling reads into contigs and aligning the contigs to a reference; (ii) aligning individual reads to the reference; (iii) assembling reads into contigs, aligning the contigs to a reference, and aligning the individual reads to the contigs; or (iv) other strategies known to be developed or known in the art. Sequence assembly can be done by methods known in the art including reference-based assemblies, de novo assemblies, assembly by alignment, or combination methods. Sequence assembly is described in U.S. Pat. No. 8,165,821; U.S. Pat. No. 7,809,509; U.S. Pat. No. 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501). SSAKE cycles through a table of reads and searches a prefix tree for the longest possible overlap between any two sequences. SSAKE clusters reads into contigs.

Generally, read assembly and analysis will proceed through the use of one or more specialized computer programs. One read assembly program is Forge Genome Assembler, written by Darren Platt and Dirk Evers and available through the SourceForge web site maintained by Geeknet (Fairfax, Va.) (see, e.g., DiGuistini et al., 2009, De novo sequence assembly of a filamentous fungus using Sanger, 454 and IIlumina sequence data, Genome Biology, 10:R94). Forge distributes its computational and memory consumption to multiple nodes, if available, and has therefore the potential to assemble large sets of reads. Forge was written in C++ using the parallel MPI library. Forge can handle mixtures of reads, e.g., Sanger, 454, and Illumina reads.

Another exemplary read assembly program known in the art is Velvet, available through the web site of the European Bioinformatics Institute (Hinxton, UK) (Zerbino & Birney, Velvet: Algorithms for de novo short read assembly using de Bruijn graphs, Genome Research 18(5):821-829). Velvet implements an approach based on de Bruijn graphs, uses information from read pairs, and implements various error correction steps.

Read assembly can be performed with the programs from the package SOAP, available through the website of Beijing Genomics Institute (Beijing, CN) or BGI Americas Corporation (Cambridge, Mass.). For example, the SOAPdenovo program implements a de Bruijn graph approach. SOAP3/GPU aligns short reads to a reference sequence.

Another read assembly program is ABySS, from Canada's Michael Smith Genome Sciences Centre (Vancouver, B.C., CA) (Simpson et al., 2009, ABySS: A parallel assembler for short read sequence data, Genome Res., 19(6):1117-23). ABySS uses the de Bruijn graph approach and runs in a parallel environment.

Read assembly can also be done by Roche's GS De Novo Assembler, known as gsAssembler or Newbler (NEW assemBLER), which is designed to assemble reads from the Roche 454 sequencer (described, e.g., in Kumar & Blaxter, 2010, Comparing de novo assemblers for 454 transcriptome data, Genomics 11:571 and Margulies 2005). Newbler accepts 454 Flx Standard reads and 454 Titanium reads as well as single and paired-end reads and optionally Sanger reads. Newbler is run on Linux, in either 32 bit or 64 bit versions. Newbler can be accessed via a command-line or a Java-based GUI interface. Additional discussion of read assembly may be found in Li et al., 2009, The Sequence alignment/map (SAM) format and SAMtools, Bioinformatics 25:2078; Lin et al., 2008, ZOOM! Zillions Of Oligos Mapped, Bioinformatics 24:2431; Li & Durbin, 2009, Fast and accurate short read alignment with Burrows-Wheeler Transform, Bioinformatics 25:1754; and Li, 2011, Improving SNP discovery by base alignment quality, Bioinformatics 27:1157. Assembled sequence reads may preferably be aligned to a reference. Methods for alignment and known in the art and may make use of a computer program that performs alignment, such as Burrows-Wheeler Aligner.

### 3. Variant Calling

Aligned or assembled sequence reads may be analyzed for the presence of variants, e.g., mutations described, or “called” as variants of a given reference. Mutation calling is described in U.S. Pub. 2013/0268474. In certain embodiments, analyzing the reads includes assembling the sequence reads and then genotyping the assembled reads.

In certain embodiments, reads are aligned to hg18 on a per-sample basis using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9):1297-1303 (aka the GATK program). High-confidence genotype calls may be defined as having depth ≧50 and strand bias score ≦0. De-barcoded fastq files are obtained as described above and partitioned by capture region (exon) using the target arm sequence as a unique key. Reads are assembled in parallel by exon using SSAKE version 3.7 with parameters “-m 30 -o 15”. The resulting contiguous sequences (contigs) can be aligned to hg18 (e.g., using BWA version 0.5.7 for long alignments with parameter “-r 1”). In some embodiments, short-read alignment is performed as described above, except that sample contigs (rather than hg18) are used as the input reference sequence. Software may be developed in Java to accurately transfer coordinate and variant data (gaps) from local sample space to global reference space for every BAM-formatted alignment. Genotyping and base-quality recalibration may be performed on the coordinate-translated BAM files using the GATK program.

In some embodiments, any or all of the steps of the invention are automated. For example, a Perl script or shell script can be written to invoke any of the various programs discussed above (see, e.g., Tisdall, Mastering Perl for Bioinformatics, O'Reilly & Associates, Inc., Sebastopol, C A 2003; Michael, R., Mastering Unix Shell Scripting, Wiley Publishing, Inc., Indianapolis, Ind. 2003). Alternatively, methods of the invention may be embodied wholly or partially in one or more dedicated programs, for example, each optionally written in a compiled language such as C++ then compiled and distributed as a binary. Methods of the invention may be implemented wholly or in part as modules within, or by invoking functionality within, existing sequence analysis platforms. In certain embodiments, methods of the invention include a number of steps that are all invoked automatically responsive to a single starting queue (e.g., one or a combination of triggering events sourced from human activity, another computer program, or a machine). Thus, the invention provides methods in which any or the steps or any combination of the steps can occur automatically responsive to a queue. Automatically generally means without intervening human input, influence, or interaction (i.e., responsive only to original or pre-queue human activity).

With continued reference to FIG. 3, mapping 323 sequence reads to a reference, by whatever strategy, may produce output such as a text file or an XML file containing sequence data such as a sequence of the nucleic acid aligned to a sequence of the reference genome. In certain embodiments mapping reads to a reference produces results stored in SAM or BAM file (e.g., as shown in FIG. 3) and such results may contain coordinates or a string describing one or more mutations in the subject nucleic acid relative to the reference genome. Alignment strings known in the art include Simple UnGapped Alignment Report (SUGAR), Verbose Useful Labeled Gapped Alignment Report (VULGAR), and Compact Idiosyncratic Gapped Alignment Report (CIGAR). See Ning et al., 2001, SSAHA: A fast search method for large DNA database, Genome Research 11(10):1725-9. These strings are implemented, for example, in the Exonerate sequence alignment software from the European Bioinformatics Institute (Hinxton, UK).

In some embodiments, a sequence alignment is produced—such as, for example, a sequence alignment map (SAM) or binary alignment map (BAM) file 329—comprising a CIGAR string (the SAM format is described, e.g., in Li, et al., The Sequence Alignment/Map format and SAMtools, Bioinformatics, 2009, 25(16):2078-9). In some embodiments, CIGAR displays or includes gapped alignments one-per-line. CIGAR is a compressed pairwise alignment format reported as a CIGAR string. A CIGAR string is useful for representing long (e.g. genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The CIGAR string defines the sequence of matches/mismatches and deletions (or gaps). For example, the CIGAR string 2MD3M2D2M will mean that the alignment contains 2 matches, 1 deletion (number 1 is omitted in order to save space), 3 matches, 2 deletions and 2 matches. In general, for carrier screening or other assays such as the NGS workflow depicted in FIG. 3, sequencing results will be used in genotyping.

Output from mapping may be stored in a SAM or BAM file, in a variant call format (VCF) file 335, or other format. In an illustrative embodiment, output is stored in a VCF file. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described in Danecek et al., 2011, The variant call format and VCF tools, Bioinformatics 27(15):2156-2158.

The data contained in a VCF file represents the variants, or mutations, that are found in the nucleic acid that was obtained from the sample from the patient and sequenced. In its original sense, mutation refers to a change in genetic information and has come to refer to the present genotype that results from a mutation. As is known in the art, mutations include different types of mutations such as substitutions, insertions or deletions (INDELs), translocations, inversions, chromosomal abnormalities, and others. By convention in some contexts where two or more versions of genetic information or alleles are known, the one thought to have the predominant frequency in the population is denoted the wild type and the other(s) are referred to as mutation(s). In general in some contexts an absolute allele frequency is not determined (i.e., not every human on the planet is genotyped) but allele frequency refers to a calculated probable allele frequency based on sampling and known statistical methods and often an allele frequency is reported in terms of a certain population such as humans of a certain ethnicity. Variant can be taken to be roughly synonymous to mutation but referring to a genotype being described in comparison or with reference to a reference genotype or genome. For example as used in bioinformatics variant describes a genotype feature in comparison to a reference such as the human genome (e.g., hg18 or hg19 which may be taken as a wild type). Methods described herein generate data representing one or more mutations, or “variant calls.”

A description of a mutation may be provided according to a systematic nomenclature. For example, a variant can be described by a systematic comparison to a specified reference which is assumed to be unchanging and identified by a unique label such as a name or accession number. For a given gene, coding region, or open reading frame, the A of the ATG start codon is denoted nucleotide +1 and the nucleotide 5′ to +1 is −1 (there is no zero). A lowercase g, c, or m prefix, set off by a period, indicates genomic DNA, cDNA, or mitochondrial DNA, respectively.

A systematic name can be used to describe a number of variant types including, for example, substitutions, deletions, insertions, and variable copy numbers. A substitution name starts with a number followed by a “from to” markup. Thus, 199A>G shows that at position 199 of the reference sequence, A is replaced by a G. A deletion is shown by “del” after the number. Thus 223delT shows the deletion of T at nt 223 and 997-999del shows the deletion of three nucleotides (alternatively, this mutation can be denoted as 997-999delTTC). In short tandem repeats, the 3′ nt is arbitrarily assigned; e.g. a TG deletion is designated 1997-1998delTG or 1997-1998del (where 1997 is the first T before C). Insertions are shown by ins after an interval. Thus 200-201insT denotes that T was inserted between nts 200 and 201. Variable short repeats appear as 997(GT)N-N′. Here, 997 is the first nucleotide of the dinucleotide GT, which is repeated N to N′ times in the population.

Variants in introns can use the intron number with a positive number indicating a distance from the G of the invariant donor GU or a negative number indicating a distance from an invariant G of the acceptor site AG. Thus, IVS3+1C>T shows a C to T substitution at nt+1 of intron 3. In any case, cDNA nucleotide numbering may be used to show the location of the mutation, for example, in an intron. Thus, c.1999+1C>T denotes the C to T substitution at nt+1 after nucleotide 1997 of the cDNA. Similarly, c.1997−2A>C shows the A to C substitution at nt−2 upstream of nucleotide 1997 of the cDNA. When the full length genomic sequence is known, the mutation can also be designated by the nt number of the reference sequence.

Relative to a reference, a patient's genome may vary by more than one mutation, or by a complex mutation that is describable by more than one character string or systematic name. The invention further provides systems and methods for describing more than one variant using a systematic name. For example, two mutations in the same allele can be listed within brackets as follows: [1997G>T; 2001A>C]. Systematic nomenclature is discussed in den Dunnen & Antonarakis, 2003, Mutation Nomenclature, Curr Prot Hum Genet 7.13.1-7.13.8 as well as in Antonarakis and the Nomenclature Working Group, 1998, Recommendations for a nomenclature system for human gene mutations, Human Mutation 11:1-3. Variant detection can include using a system of the invention. For one suitable system, see U.S. Pat. No. 8,812,422, incorporated by reference. Variants may be named according to HGVS-recommended nomenclature or any other systematic mutation nomenclature. Mutations in the database (e.g., for comparison to sequencing results from a MIP carrier screening) may be classified. Classification criteria described here apply to recessive Mendelian disorders and highly penetrant variants with relatively large effects. Classification criteria may follow recommendations in the literature: Richards et al., ACMG recommendations for standards for interpretation and reporting of sequence variations: Revisions 2007, Genet Med 2008, 10:294-300; Maddalena et al., Technical standards and guidelines: molecular genetic testing for ultra-rare disorders, Genet Med 2005, 7:571-83; and Strom C M, Mutation detection, interpretation, and applications in the clinical laboratory setting, Mutat Res 2005, 573:160-7, each incorporated by reference. Classification may be based on any suitable combination of sequence-based evidence (e.g., being a truncating mutation), experimental evidence, or genetic evidence (e.g., classified as pathogenic based on genetic evidence if it was a founder variant, or if there was statistical evidence showing the variant was significantly more frequent in affected individuals than in controls; see MacArthur et al., Guidelines for investigating causality of sequence variants in human disease, Nature 2014, 508:469-76). For methods suitable for use in detection of variants detectable by the standard NGS protocol, see Umbarger et al., Next-generation carrier screening, Genet Med 2014, 16:132-40 and Hallam et al., Validation for Clinical Use of, and Initial Clinical Experience with, a Novel Approach to Population-Based Carrier Screening using High-Throughput, Next-Generation DNA Sequencing, J Mol Diagn 2014, 16:180-9, both incorporated by reference.

Any suitable gene may be screened using methods of the invention. In a preferred embodiments, methods of the invention are used to screen for recessive Mendelian disorders. Certain genetic disorders and their associated genes that may be screened using methods of the invention include Canavan disease (ASPA), cystic fibrosis (CFTR), glycogen storage disorder type 1a (G6PC), Niemann-Pick disease (SMPD1), Tay-Sachs disease (HEXA), Bloom syndrome (BLM), Fanconi anemia C (FANCC), familial Hyperinsulinism (ABCC8), maple syrup urine disease type 1A (BCKDHA) and type 1B (BCKDHB), Usher syndrome type III (CLRN1), dihydrolipoamide dehydrogenase deficiency (DLD), familial dysautonomia (IKBKAP), mucolipidosis type IV (MCOLN1), and Usher syndrome type 1F (PCDH15).

### 4. Functional Assessment

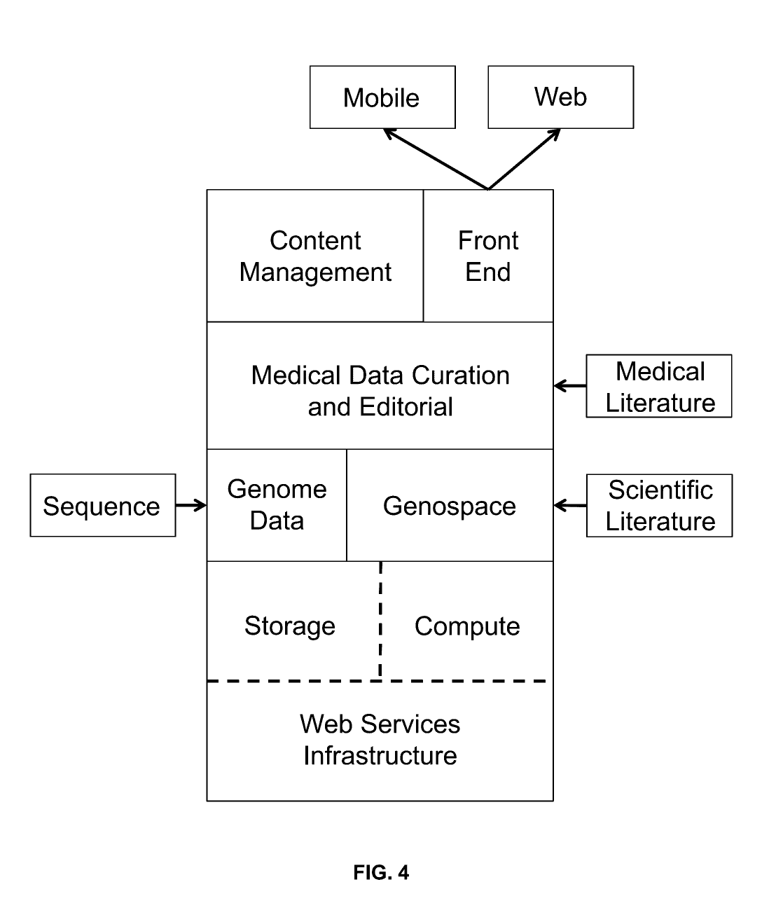

FIG. 4 illustrates a platform architecture for implementing methods of the invention. The platform may be built on a web services infrastructure such as, for example, Amazon Web Services (AWS). The services infrastructure may provide storage and compute modules or functionality. The raw sequence data is brought in and through assembly or variant calling is taken as the patient's genome data. The scientific literature at large integrates by means of a genomic platform for biomedical analysis such as, for example, the service sold under the name GENOSPACE by Genospace (Cambridge, Mass.).

Queries against the genomic platform can provide functional information about a variant. Such information may include what gene it lies within, if any; is the variant inside or outside of an intron, exon, other feature, or does it span a boundary; does the variant lie within an open reading frame; or does the variant create a frameshift or missense or nonsense mutation or premature stop codon or silent mutation. Functional assessment 117 may proceed using tools such as Genospace, Broad Inst., Signifikance, etc., assess a functional impact of a variant.

For patient reporting or notification, systems and methods of the invention may be used to retrieve medical/clinical information from an outside database. The outside database is preferably a clinical decision support system such as UP2DATE by Wolters-Kluwer. Any suitable clinical decision support resources may be included in the outside database that is queried by the system. Other suitable resources include the medical reference resource sold under the name EPOCRATES by Athena Health (Watertown, Mass.). Other clinical decision support (CDS) resources that may be accessed may include the PREDICT (Pharmacogenomic Resource for Enhanced Decisions in Care and Treatment) project, the CLIPMERGE (Clinical Implementation of Personalized Medicine through Electronic Health Records and Genomics) program, and the SMART (Substitutable Medical Apps Reusable Technologies) Genomics Adviser. The PREDICT project uses CDS functionality of an electronic record, StarPanel, to provide active CDS. PREDICT is currently designed to include both preemptive testing and “just in time,” indication-based testing. To date, >11,000 individuals have been tested in PREDICT using the Illumina ADME platform, which includes 34 genes and 184 variants. Genomics Adviser is available as a stand-alone external CDS technology or it can be integrated with other applications.

The outside database may represent a distillation of the medical literature at large. Specifically, a curated database is used wherein curators work from the medical literature to keep the database up to date. Typically, the outside database will include medical data and metadata, where the medical data represents the intended content (e.g., accessible by a subscriber by opening an SQL handle) and the metadata represents internal information such as a revision history. In a preferred embodiment, an outside database is used in which each update is labeled with metadata that characterizes the update. For example, the metadata may identify the update as one or more of: correct a typo; new SNP added; clinical trial initiated; new primers published; mutation description transcluded from OMIM; author list changed. A front end module provides a web- or mobile-based interface to users.

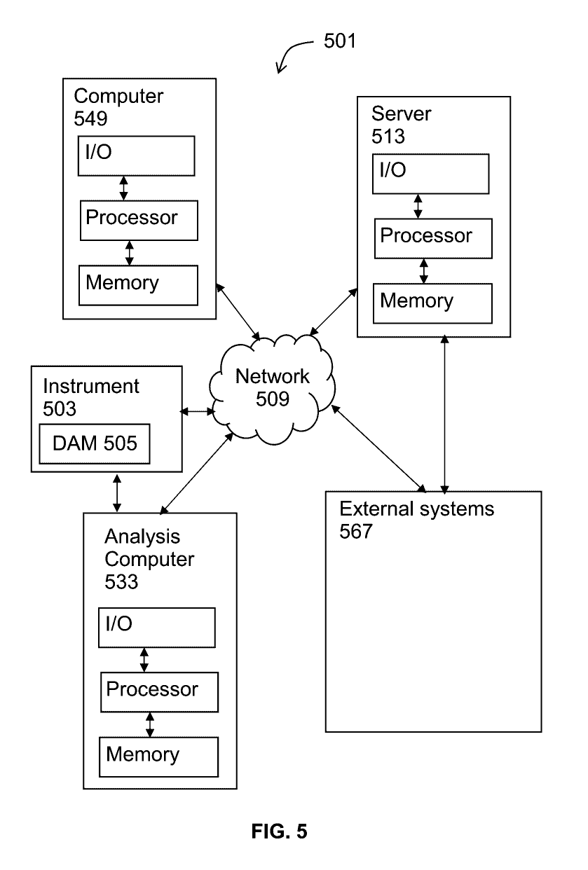

FIG. 5 gives a diagram of a system 501 according to embodiments of the invention. System 501 may include an analysis instrument 503 which may be, for example, a sequencing instrument (e.g., a HiSeq 2500 or a MiSeq by Illumina). Instrument 503 includes a data acquisition module 505 to obtain results data such as sequence read data. Instrument 503 may optionally include or be operably coupled to its own, e.g., dedicated, analysis computer 533 (including an input/output mechanism, one or more processor, and memory). Additionally or alternatively, instrument 503 may be operably coupled to a server 513 or computer 549 (e.g., laptop, desktop, or tablet) via a network 509.

Computer 549 includes one or more processors and memory as well as an input/output mechanism. Where methods of the invention employ a client/server architecture, steps of methods of the invention may be performed using the server 513, which includes one or more of processors and memory, capable of obtaining data, instructions, etc., or providing results via an interface module or providing results as a file. The server 513 may be provided by a single or multiple computer devices, such as the rack-mounted computers sold under the trademark BLADE by Hitachi. The server 513 may be provided as a set of servers located on or off-site or both. The server 513 may be owned or provided as a service. The server 513 may be provided wholly or in-part as a cloud-based resources such as Amazon Web Services or Google. The inclusion of cloud resources may be beneficial as the available hardware scales up and down immediately with demand. The actual processors—the specific silicon chips—performing a computation task can change arbitrarily as information processing scales up or down. In a preferred embodiment, the server 513 includes one or a plurality of local server boxes working in conjunction with a cloud resource (where local means not-cloud and includes or or off-site). The server 513 may be engaged over the network 509 by the computer 549 and either or both may engage the outside database 567.

In system 501, each computer preferably includes at least one processor coupled to a memory and at least one input/output (I/O) mechanism.

A processor will generally include a chip, such as a single core or multi-core chip, to provide a central processing unit (CPU). A process may be provided by a chip from Intel or AMD.

Memory can include one or more machine-readable devices on which is stored one or more sets of instructions (e.g., software) which, when executed by the processor(s) of any one of the disclosed computers can accomplish some or all of the methodologies or functions described herein. The software may also reside, completely or at least partially, within the main memory and/or within the processor during execution thereof by the computer system. Preferably, each computer includes a non-transitory memory such as a solid state drive, flash drive, disk drive, hard drive, etc. While the machine-readable devices can in an exemplary embodiment be a single medium, the term “machine-readable device” should be taken to include a single medium or multiple media (e.g., a centralized or distributed database, and/or associated caches and servers) that store the one or more sets of instructions and/or data. These terms shall also be taken to include any medium or media that are capable of storing, encoding, or holding a set of instructions for execution by the machine and that cause the machine to perform any one or more of the methodologies of the present invention. These terms shall accordingly be taken to include, but not be limited to one or more solid-state memories (e.g., subscriber identity module (SIM) card, secure digital card (SD card), micro SD card, or solid-state drive (SSD)), optical and magnetic media, and/or any other tangible storage medium or media.

A computer of the invention will generally include one or more I/O device such as, for example, one or more of a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Any of the software can be physically located at various positions, including being distributed such that portions of the functions are implemented at different physical locations.

System 501 or components of system 501 may be used to perform methods described herein. Instructions for any method step may be stored in memory and a processor may execute those instructions. System 501 or components of system 501 may be used for the analysis of genomic sequences or sequence reads (e.g., detecting deletions and variant calling).

The system 501 engages the external database or databases 567 to obtain medical information. A first aspect of medical information is disease association and another important aspect involves actionable clinical information.

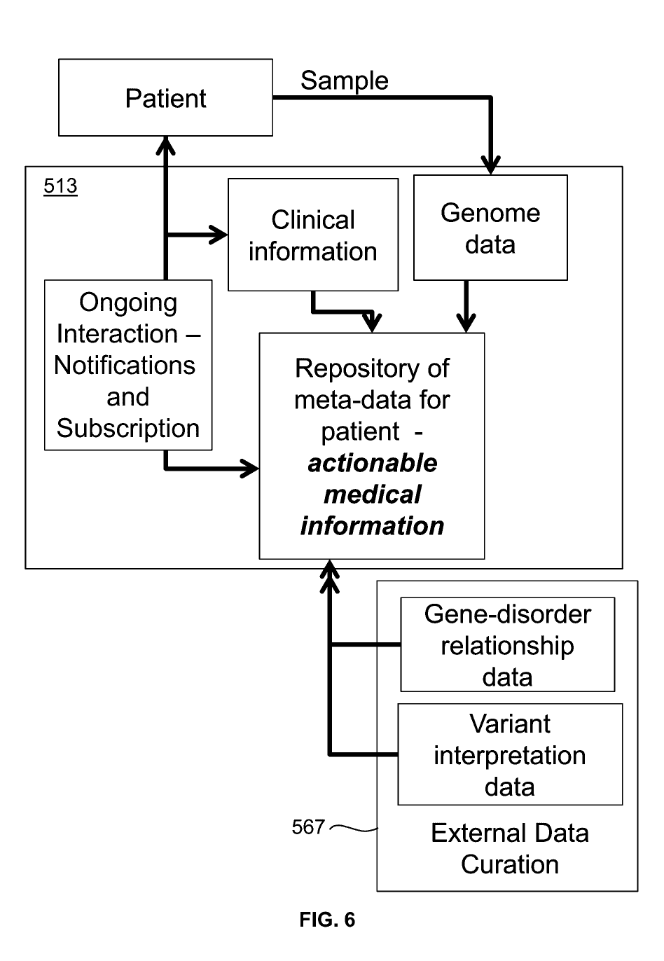

FIG. 6 diagrams a workflow for the medical information. The system provides a repository of patient information and meta-data, which includes clinical information, genome data, and patient subscription information. The system 501 can query curated external databases 567 for disease association and reporting information.

### 5. Data Retrieval

In disease association 121, variants may be associated with a disease and any additional information. For example, information may be obtained from a such a source as Genospace, OMIM, or Rancho Biosciences through a systematic and semantically-controlled combination of manual and automated curation. Typically, disease association provides, for a variant, any disease known to be associated with that variant. For many variants (e.g., hundreds to thousands), the disease associations may be provided by existing internal databases. For example, functional assessment may locate a SNP within a cystic fibrosis transmembrane receptor and that SNP may already be tracked in an internal database within the server 513 (see, e.g., U.S. Pat. No. 8,812,422, incorporated by reference) as associated with the disease cystic fibrosis. On top of disease association, systems of the invention can include, in provided patient reports, actionable medical information.

The medical text can be provided by querying an outside source such as a clinical decision support resource as the outside database. One suitable product is the clinical decision support resource offered under the trademark UP2DATE by Wolters-Kluwer. Systems and methods of the invention use automated access to structured, actionable medical information for specific diseases from the outside database and provide for custom integration of updates based on new “tagged content” from the outside database. Systems of the invention implement automated access to structured, actionable medical information for specific diseases. Custom integration of updates may be based on new “tagged content” from the outside database.

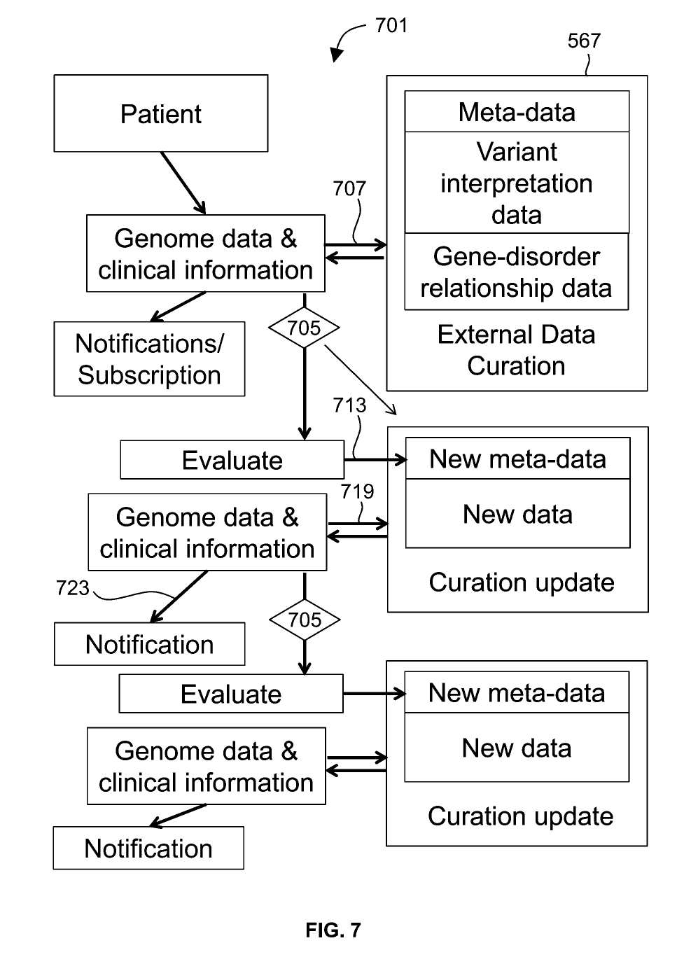

FIG. 7 diagrams a method 701 for determining whether to notify a user of the availability of an updated report. Initially, the system may provide a report for a user that includes an identity of the patient, the variant call, and the clinical information on the variant and the also later provide an updated reported with the updated clinical information. The initial report may be provided by querying 707 the outside database 567 for curated variant interpretation data.

Going forward, the system 513 determines whether an update to the clinical information has been published. This may be done by simply comparing the present information to the information that was last used in a report for the patient about the mutation. If there has been an updated, the system evaluates 713 whether the update meets predetermined criteria for significance and notifies 723 a user of updated clinical information meeting the predetermined criteria for significance. The system may be used to compose a new report by querying 719 the outside database 567, specifically the updated data, and provide the new report, which preferably includes new clinical information associated with the variant. The new clinical information associated may include one or more of a functional information, a disease association, and medical information. Preferably, the new clinical information includes updated information about an association of a variant in the sequence data with a medical condition, a prognosis, a treatment regimen, or a propensity for disease.

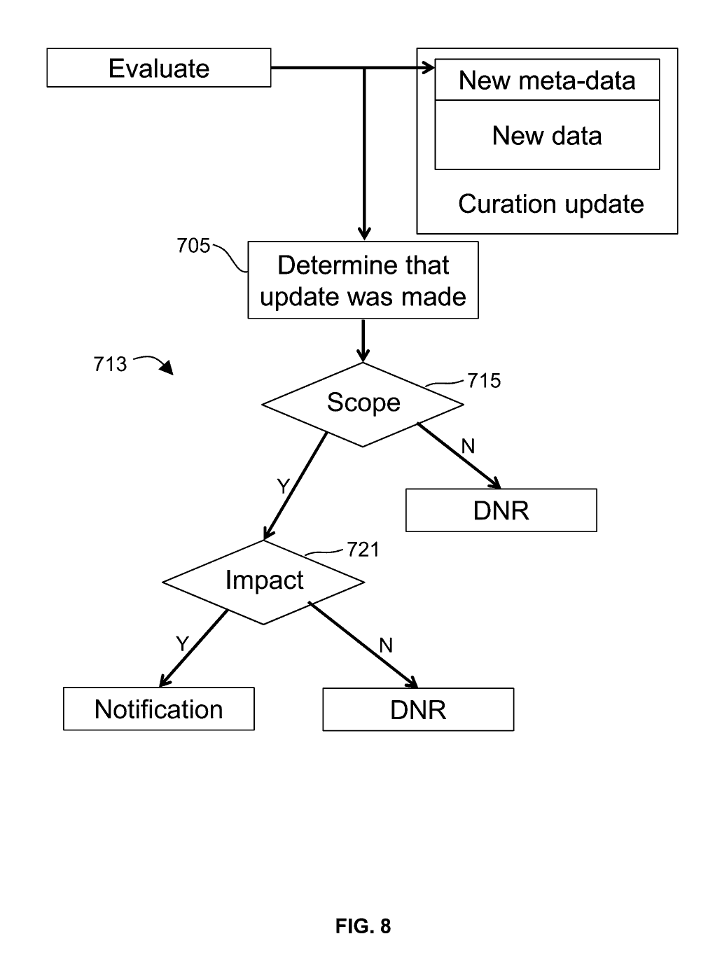

FIG. 8 is a flowchart for determining a significance of an update. The evaluating step comprises reading metadata entered into said database. The metadata may include such information as a source of the update, a date of the update, and the predetermined criteria.

The evaluation of the significance of the new information can take into account both the scope of the change and the impact to the particular patient. Thus the evaluation may include a scope assessment 715 and an impact assessment 721.

The scope assessment 715 looks at the substance of the update. Typically in the outside database, the curators will tag updates with metadata that characterizes the update. The outside database may provide a defined schema for the metadata tags and system 513 may be programmed to read the metadata for certain predefined tags that indicate the scope or substance of the update. Exemplary tags that may be read in the scope assessment, and whether the scope assessment results in a “Yes, proceed” or a “No, do not report”, may include, for example: <minor edit></minor edit> “N”; <new disease></new disease> “Y”; <accession number assigned></accession number assigned> “N”; and <FDA treatment approval></FDA treatment approval> “Y”.

The impact assessment 721 queries whether an update has applicability to a patient with a particular mutation. For example, where a disease phenotype is known to require an indel proximal to a SNP, for a patient with the SNP but not the indel, new medical information about the SNP may be determined to not be impactful to that patient. Thus by means of the scope assessment 715 and the impact assessment 721 some updates may be deemed trivial and ignored, for example, where a minor change is documented in incidence of a disease in some demographic. Additionally, updates need not trigger a notification if not relevant to the patient, for example, a where a SNP is linked to prostate cancer a female patient may not be given an urgent notification.

The update and notification steps may be performed once, multiple times, regularly, periodically, on-demand, or according to any other desired schedule (e.g., the determining, evaluating, and notifying steps are performed a plurality of times for a plurality of different updates over a period of at least a week).

Systems and methods of the invention provide a user interface 131 via, for example, a mobile app or a desktop web app. The user interface provides personalized access to updated data. Physicians or genetic counselors or their patients may receive alerts generated by curated updates of relevant information automatically pushed out to the users.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for updating informative content of genomic information, the method comprising:
obtaining sequence data from a sample from a patient;
inputting the sequence data into a computer system having a processor coupled to a tangible memory subsystem;
retrieving from a database clinical information on at least one variant in the sequence data;
associating the clinical information with the variant in the memory subsystem;
determining whether an update to the clinical information has been published;
evaluating whether the update meets predetermined criteria for significance; and
notifying a user of updated clinical information meeting the predetermined criteria for significance.

2. The method of claim 2, wherein the database is a curated database on a remote computer system.

3. The method of claim 2, wherein the evaluating step comprises reading metadata entered into the database.

4. The method of claim 3, wherein the metadata identifies at least one of a source of the update, a date of the update, and the predetermined criteria.

5. The method of claim 3, wherein obtaining the sequence data comprises sequencing nucleic acid from the sample to obtain a plurality of sequence reads.

6. The method of claim 5, further comprising mapping the sequence reads to a genomic reference to identify the at least one variant and storing the at least one variant in the memory subsystem as a variant call prior to retrieving the information on the variant.

7. The method of claim 6, further comprising providing a report for a user that includes an identity of the patient, the variant call, and the clinical information on the variant; and
later providing an updated reported with the updated clinical information.

8. The method of claim 7, wherein the clinical information associated with the variant in the memory subsystem includes one or more of a functional information, a disease association, and medical information.

9. The method of claim 1, further comprising providing a report to a user prior to determining whether the update has been published, wherein the report comprises the clinical information associated with the variant and an identity of the patient.

10. The method of claim 9, wherein the determining, evaluating, and notifying steps are performed a plurality of times for a plurality of different updates over a period of at least a week.

11. The method of claim 10, wherein the clinical information comprises an association of a variant in the sequence data with a medical condition, a prognosis, a treatment regimen, or a propensity for disease.

12. The method of claim 11, wherein at least a portion of the computer system is provided by a cloud-based system in which another processor may be substituted for the processor without interfering with operation of the method.

13. The method of claim 10, wherein notifying the user of the updated clinical information comprises sending an alert from the computer system to a user computer device.

14. The method of claim 10, wherein notifying the user of the updated clinical information comprises causing the alert to be displayed on a mobile or web interface on the user computer device.

15. The method of claim 14, wherein obtaining the sequence data comprises sequencing nucleic acid from the sample to obtain a plurality of sequence reads, the method further comprising:
mapping the sequence reads to a genomic reference to identify the at least one variant;
storing the at least one variant in the memory subsystem as a variant call prior to retrieving the information on the variant;
providing a report for a user that includes an identity of the patient, the variant call, and the clinical information on the variant; and
later providing an updated reported with the updated clinical information.

16. A system for updating informative content of genomic information, the system comprising a processor coupled to a tangible memory subsystem storing instructions that when executed by the processor cause the system to:
obtain sequence data from a sample from a patient;
retrieve from a database clinical information on at least one variant in the sequence data;
associate the clinical information with the variant in the memory subsystem;
determine whether an update to the clinical information has been published;
evaluate whether the update meets predetermined criteria for significance; and
notify a user of updated clinical information meeting the predetermined criteria for significance.

17. The system of claim 16, wherein the database is a curated database on a remote computer system.

18. The system of claim 17, wherein the evaluating step comprises reading metadata entered into said database.

19. The system of claim 18, wherein the metadata identifies at least one of a source of the update, a date of the update, and the predetermined criteria.

20. The system of claim 15, further operable to map the sequence data to a genomic reference to identify the at least one variant and store the at least one variant in the memory subsystem as a variant call prior to retrieving the information on the variant.

21. The system of claim 20, further operable to provide a report for a user that includes an identity of the patient, the variant call, and the clinical information on the variant; and
later provide an updated reported with the updated clinical information.

22. The system of claim 21, wherein the clinical information associated with the variant in the memory subsystem includes one or more of a functional information, a disease association, and medical information.

23. The system of claim 21, further operable to provide a report to a user prior to determining whether the update has been published, wherein the report comprises the clinical information associated with the variant and an identity of the patient.

24. The system of claim 23, wherein the system performs the determining, evaluating, and notifying steps a plurality of times for a plurality of different updates over a period of at least a week.

25. The system of claim 24, wherein the clinical information comprises an association of a variant in the sequence data with a medical condition, a prognosis, a treatment regimen, or a propensity for disease.

26. The system of claim 26, wherein at least a portion of the system is provided by a cloud-based system in which another processor may be substituted for the processor without interfering with operation of the method.
